(19) United States
(12) Patent Application Publication
(10) Pub. No.: US 2004/0009972 A1
(43) Pub. Date: Jan. 15, 2004

(54) Benzodiazepine inhibitors of mitochondial F1F0 ATP hydrolase and methods of inhibiting F1F0 ATP hydrolase

(76) Inventors: Charles Z. Ding, Plano, TX (US); Lawrence G. Hamann, Cherry Hill, NJ (US); Philip D. Stein, Pennington, NJ (US); Andrew T. Pudzianowski, Yardley, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/461,736

(22) Filed: Jun. 13, 2003

Related U.S. Application Data
(60) Provisional application No. 60/389,213, filed on Jun. 17, 2002.

(51) Int. Cl. A61K 31/5513; C07D 243/14

(52) U.S. Cl. 514/221; 540/573

(57) ABSTRACT

Compounds having the formula (I),

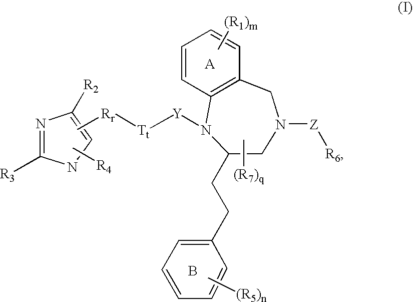

are useful as inhibitors of mitochondrial F₁F₀ ATP hydrolase, wherein R₁, R₅ and R₇ are optional substituents, R₂, R₃ and R₄ are hydrogen, alkyl, or substituted alkyl, or comprise a bond to R, T or Y; Z and Y are selected from C(═O), —CO₂—, —SO₂—, —CH₂—, —CH₂C(═O)—, and —C(═O)C(═O)—, or Z may be absent; R and T are CH₂—, —C(═O)—, or —CH[(CH₂)ₚ(Q)]—, wherein Q is NR₁₀R₁₁, OR₁₀ or CN and p is 0, 1 or 2; R₆ is alkyl, alkenyl, substituted alkyl, substituted alkenyl, aryl, cycloalkyl, heterocyclo, or heteroaryl; R₁₀ and R₁₁ are hydrogen, alkyl, or substituted alkyl; and r and t are 0 or 1.

This application claims the benefit of priority from U.S. provisional application serial No. 60/389,213 filed Jun. 17, 2002, which is incorporated herein by reference.

### FIELD OF THE INVENTION

This invention relates to benzodiazepine compounds that inhibit mitochondrial FIFO ATP hydrolase and are useful for treating ischemia-related diseases. The invention further pertains to methods of treating conditions associated with depleted levels of adenosine triphosphate (ATP) due to hydrolysis by mitochondrial F₁F₀ ATPase.

### BACKGROUND OF THE INVENTION

Ischemic heart disease is a common and serious health problem. Every year, large numbers of patients die from ischemic heart disease and its complications. Many others experience acute myocardial infarcation, congestive heart failure, cardiac arrhythmias, or other disorders.

Myocardial ischemia exists when the heart tissue experiences a demand for oxygen and substrates that exceed the supply. Imbalances between oxygen supply and demand span a large range, and thus, there are various syndromes and biochemical pathways involved in the pathogenesis of ischemia, e.g., from low-grade to severe ischemic conditions. For example, chronic stable angina pectoris is a low-grade condition, in which the resting coronary blood flow may be normal but the blood flow reserve is insufficient to meet an increased energy demand. In more extreme situations, the ischemic muscle can develop an impaired contractile function and potential to generate arrhythmias. Major consequences of myocardial ischemia include mechanical and electrical dysfunction, muscle cell damage, and development of necrosis. Acute ischemic events may develop where there is coronary atherosclerosis. Ultimately, if the ischemia is sufficiently severe there will be an immediate reduction (or cessation) of contractile function in the heart.

The impairment of contractile function in ischemic muscle is associated with mitochondrial levels of adenosine triphosphate (ATP) and adenosine triphosphatases (ATPases). ATPases are enzymes that typically catalyze the hydrolysis of ATP, the main energy currency in cells, to adenosine monophosphate (AMP) or adenosine diphosphate (ADP), plus phosphate ions and energy. The contractile function of the heart is regulated by the transport of calcium, sodium, and potassium ions, which in turn is modulated by ATP and ATPases. More particularly, intracellular ATP is split by Na+,K+ ATPase, an enzyme that is responsible for maintaining a gradient of sodium and potassium ions across the cell membrane. The splitting of ATP by Na+,K+ATPase releases the energy needed to transport K+ and Na+ ions against concentration gradients. This enables the existence of a resting potential in the membrane (i.e, Na+ out, K+ in) which initiates the contractile response. Contraction is triggered by Na/Ca exchange and Ca²⁺ transport, the energy for which is generated by the hydrolysis of ATP by Ca²⁺ ATPase.

To maintain homeostasis, the cells' supply of ATP must be replenished as it is consumed (e.g., with muscle contraction). During the steady state, the rate of ATP synthesis needs to be closely matched to its rate of consumption. Arguably, the most important ATPase is the mitochondrial F₁F₀-ATPase. Unlike other ATPases which function typically to hydrolyze ATP and release energy, the F₁F₀-ATPase has both hydrolytic and synthetic states. As “ATP synthase”, the mitochondrial F₁F₀-ATPase catalyzes the production of ATP via oxidative phosphorylation of ADP and Pᵢ. Thus, F₁F₀-ATPase is responsible for producing the cell's main energy source, ATP. In normoxic conditions, mitochondrial F₁F₀-ATPase modulates this ATP production via its two units, the F₁ and F₀ complexes. F₀ is the inner membrane domain, and F₁ is a catalytic domain consisting of five subunits (αβχδε—the catalytic site is on the β unit), that protrude from the F₀ domain into the mitochondrial matrix. When sufficient levels of oxygen are present, electrons from ATPase substrates are transferred to oxygen, and, protons are transported out of the mithcondrial matrix. This proton/electron transport creates an electrochemical proton gradient across the mitochondrial membrane and through the F₀ domain which drives the F₁ domain to synthesize ATP.

In ischemic conditions, however, this electrochemical gradient collapses, and F₁F₀-ATPase switches to its hydrolytic state. This hydrolysis of ATP seems to serve no useful purpose. Also, as F₁F₀-ATPase operates in its hydrolytic state there is a down-regulation of F₁F₀-ATP synthase. F₁F₀-ATP synthase activities in vesicles from ischemic muscle typically are substantially (up to 50-80%) less than those of control muscle. A native peptide called IF₁ inhibitor protein (or IF₁) may be bound to the F₁ unit under ischemic conditions to inhibit the ATP hydrolase activity of the enzyme; however, IF₁ is highly pH dependent and in severe conditions can provide only a modicum of control. The conversion of F₁F₀-ATP synthase to F₁F₀-ATP hydrolase is reversible, as addition of substrate and oxygen to the mitochondria of ischemic muscle can reactivate the F₁F₀-ATPase and ATP levels to control levels.

As may be appreciated, in ischemic conditions the activity of F₁F₀-ATPase produces a futile cycling and waste of ATP. It is believed that this depletion of ATP and/or ATP synthase may suppress the Na+K+ pump to increase cardiac contractility, vasoconstriction, sensitivity to vasoactive agents, and arterial blood pressure. Several inhibitors of F₁F₀-ATPase have been described, including efrapeptin, oligomycin, autovertin B, and azide. Oligomycin targets F₀ and reportedly postpones cell injury by preserving ATP during ischemia. However, the only known inhibitors of F₁F₀-ATPase are large proteins or peptides which are not orally bioavailable.

The instant invention provides benzodiazapine compounds that are surprisingly potent inhibitors of F₁F₀-ATP hydrolase. The compounds of the present invention are useful in treating or preventing conditions associated with ischemia, particularly myocardial ischemia and associated conditions, such as muscle cell damage, necrosis, and cardiac arrhythmias. Also, in view of their inhibitory activity, the inventive compounds may be used to treat cancer and tumor growth. Benzodiazepine compounds for use as farnesyl transferase inhibitors are described and claimed in U.S. Pat. No. 6,011,029 which is assigned to the present assignee.

Each of the patents, patent applications and publications referred to in this application are incorporated herein by reference.

### SUMMARY OF THE INVENTION

The invention is directed to compounds useful in inhibiting mitochondrial F₁F₀-ATP hydrolase having the formula (I):

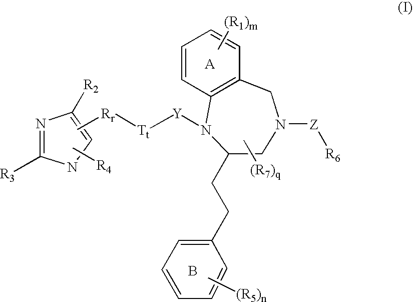

and pharmaceutically-acceptable salts, hydrates, or prodrugs thereof, wherein:

R₁ and R₅ are attached to any available carbon atom of phenyl rings A and B, respectively, and at each occurrence are independently selected from alkyl, substituted alkyl, halogen, cyano, nitro, OR₈, NR₈R₉, C(═O)R₈, CO₂R₈, C(═O)NR₈R₉, NR₈C(═O)R₉, NR₈C(═O)OR₉, S(O)ₒR₉, NR₈SO₂R₉, SO₂NR₈R₉, cycloalkyl, heterocycle, aryl, and heteroaryl, and/or two of R₁ and/or two of R₅ join together to form a fused benzo ring;

R₂, R₃ and R₄ are independently selected from hydrogen, alkyl, and substituted alkyl, or one of R₂, R₃ and R₄ is a bond to R, T or Y and the other of R₂, R₃ and R₄ is selected from hydrogen, alkyl, and substituted alkyl;

Z and Y are independently selected from C(═O), —CO₂—, —SO₂—, —CH₂—, —CH₂C(═O)—, and

—C(═O)C(═O)—, or Z may be absent;

R and T are selected from —CH₂—, —C(═O)—, and —CH[(CH₂)ₚ(Q)]—, wherein Q is NR₁₀R₁₁, OR₁₀ or CN;

R₆ is selected from alkyl, alkenyl, substituted alkyl, substituted alkenyl, aryl, cycloalkyl, heterocyclo, and heteroaryl; provided that where R₂ is hydrogen, Z-R₆ together are not —SO₂-Me or

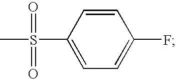

R₇ is selected from hydrogen, alkyl, substituted alkyl, alkenyl, substituted alkenyl, aminoalkyl, halogen, cyano, nitro, keto (═O), hydroxy, alkoxy, alkylthio, C(═O)H, acyl, CO₂H, alkoxycarbonyl, carbamyl, sulfonyl, sulfonamidyl, cycloalkyl, heterocycle, aryl, and heteroaryl;

R₈ and R₉ are independently selected from hydrogen, alkyl, substituted alkyl, cycloalkyl, heterocycle, aryl, and heteroaryl, or R₈ and R₉ taken together to form a heterocycle or heteroaryl, except R₉ is not hydrogen when attached to a sulfonyl group as in SO₂R₉;

R₁₀ and R₁₁ are independently selected from hydrogen, alkyl, and substituted alkyl;

m and n are independently selected from 0, 1, 2 and 3;

o, p and q are independently 0, 1 or 2; and

r and t are 0 or 1.

Also included within the scope of the invention are pharmaceutical compositions comprising one or compounds of formula (I). Further included within the invention are methods of treating ischemic conditions and/or conditions associated with depleted levels of adenosine triphosphate (ATP) and/or the activity of mitochondrial F₁F₀ ATPase.

### DETAILED DESCRIPTION OF THE INVENTION

The following are definitions of terms used in this specification. The initial definition provided for a group or term herein applies to that group or term throughout the present specification, individually or as part of another group, unless otherwise indicated.

The term “alkyl” refers to straight or branched chain hydrocarbon groups having 1 to 12 carbon atoms, preferably 1 to 8 carbon atoms. Lower alkyl groups, that is, alkyl groups of 1 to 4 carbon atoms, are most preferred.

The term “substituted alkyl” refers to an alkyl group as defined above having one, two, three, or four substituents selected from the group consisting of halogen, trifluoromethyl, alkenyl, alkynyl, nitro, cyano, keto (═O), ORₐ, SRₐ, NRₐR_{b}, NRₐSO₂, NRₐSO₂R_{c}, SO₂R_{c}, SO₂NRₐR_{b}, CO₂Rₐ, C(═O)Rₐ, C(═O)NRₐR_{b}, OC(═O)Rₐ, —OC(═O)NRₐR_{b}, NRₐC(═O)R_{b}, NRₐCO₂R_{b}, ═N—OH, ═N—O-alkyl, aryl, heteroaryl, heterocyclo and cycloalkyl, wherein Rₐ and R_{b} are selected from hydrogen, alkyl, alkenyl, cycloalkyl, heterocyclo, aryl, and heteroaryl, and R_{c} is selected from hydrogen, alkyl, cycloalkyl, heterocyclo aryl and heteroaryl. When a substituted alkyl includes an aryl, heterocyclo, heteroaryl, or cycloalkyl substituent, said ringed systems are as defined below and thus may in turn have zero to four substituents (preferably 0-2 substituents), also as defined below. When either Rₐ, R_{b} or R_{c} is an alkyl or alkenyl, said alkyl or alkenyl may optionally be substituted with 1-2 of halogen, trifluoromethyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and/or NHCO₂(alkyl).

“Alkyl” when used in conjunction with another group such as in arylalkyl refers to a substituted alkyl in which at least one of the substituents is the specifically-named group. For example, the term arylalkyl includes benzyl, or any other straight or branched chain alkyl having at least one aryl group attached at any point of the alkyl chain. As a further example, the term carbamylalkyl includes the group —(CH₂)ₙ—NH—C(═O)alkyl, wherein n is 1 to 12.

The term “alkenyl” refers to straight or branched chain hydrocarbon groups having 2 to 12 carbon atoms and at least one double bond. Alkenyl groups of 2 to 6 carbon atoms and having one double bond are most preferred.

The term “alkynyl” refers to straight or branched chain hydrocarbon groups having 2 to 12 carbon atoms and at least one triple bond. Alkynyl groups of 2 to 6 carbon atoms and having one triple bond are most preferred.

The term “alkylene” refers to bivalent straight or branched chain hydrocarbon groups having 1 to 12 carbon atoms, preferably 1 to 8 carbon atoms, e.g., {—CH₂—}ₙ, wherein n is 1 to 12, preferably 1-8. Lower alkylene groups, that is, alkylene groups of 1 to 4 carbon atoms, are most preferred. The terms “alkenylene” and “alkynylene” refer to bivalent radicals of alkenyl and alknyl groups, respectively, as defined above.

When reference is made to a substituted alkylene, alkenylene, or alkynylene group, these groups are substituted with one to four substitutents as defined above for alkyl groups. A substituted alkylene, alkenylene, or alkynylene may have a ringed substituent attached in a spiro fashion as in

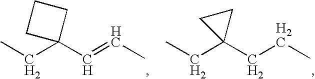

and so forth.

The term “alkoxy” refers to an alkyl, alkenyl, or substituted alkyl or alkenyl group bonded through an oxygen atom (—O—). For example, the term “alkoxy” includes the groups —O-C₁₋₁₂alkyl, —O-C₂₋₈alkenyl, —S-CH₂aryl, and so forth.

The term “alkylthio” refers to an alkyl or alkenyl or substituted alkyl or alkenyl group bonded through a sulfur (—S—) atom. For example, the term “alkylthio” includes the groups —S—(CH₂)CH₃, —S—CH₂aryl, etc.

The term “alkylamino” refers to an alkyl or alkenyl or substituted alkyl or alkenyl group bonded through a nitrogen (—NR′—) group. For example, the term “aminoalkyl” includes the groups —NR′-C₁₋₁₂alkyl and —NR′—CH₂-aryl, etc. (where R′ is hydrogen, alkyl or substituted alkyl as defined above.) “Amino” refers to the group —NH₂.

When a subscript is used as in C₁₋₈alkyl, the subscript refers to the number of carbon atoms the group may contain. Zero when used in a subscript denotes a bond, e.g., C₀₋₄alkyl refers to a bond or an alkyl of 1 to 4 carbon atoms. When used with alkoxy, thioalkyl, or alkylamino (or aminoalkyl), a subscript refers to the number of carbon atoms that the group may contain in addition to heteroatoms. Thus, for example, monovalent C₁₋₂alkylamino includes the groups —NH—CH₃, —NH—CH₂—CH₃, and —N—(CH₃)₂. A lower aminoalkyl comprises an aminoalkyl having one to four carbon atoms.

The alkoxy, thioalkyl, or aminoalkyl groups may be monovalent or bivalent. By “monovalent” it is meant that the group has a valency (i.e., power to combine with another group), of one, and by “bivalent” it is meant that the group has a valency of two. For example, a monovalent alkoxy includes groups such as —O-C₁₋₁₂alkyl, whereas a bivalent alkoxy includes groups such as —O-C₁₋₁₂alkylene-, etc.

The term “acyl” refers to a carbonyl

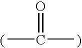

linked to an organic group i.e.,

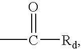

wherein R_{d} may be selected from alkyl, alkenyl, substituted alkyl, substituted alkenyl, aryl, heterocyclo, cycloalkyl, or heteroaryl, as defined herein.

The term “alkoxycarbonyl” refers to a group having a carboxy or ester group

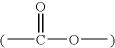

linked to an organic radical, i.e.,

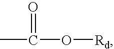

wherein R_{d} is as defined above for acyl.

The term “carbamyl” refers to a functional group in which a nitrogen atom is directly bonded to a carbonyl, i.e., as in —NRₑC(═O)R_{f} or —C(═O)NRₑR_{f}, wherein Rₑ and R_{f} can be hydrogen, alkyl, substituted alkyl, alkenyl, substituted alkenyl, alkoxy, cycloalkyl, aryl, heterocyclo, or heteroaryl, or they may join to form a ring.

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF₃.

The term “sulfonyl” refers to a sulphoxide group (i.e., —S(O)₁₋₂) linked to an organic radical R_{c}, as defined above.

The term “sulfonamidyl” or “sulfonamido” refers to the group —S(O)₂NRₑR_{f}, wherein Rₑ and R_{f} are as defined above. Preferably when one of Rₑ and R_{f} is optionally substituted heteroaryl or heterocycle (as defined below), the other of Rₑ and R_{f} is hydrogen, alkyl, or substituted alkeyl.

The term “cycloalkyl” refers to fully saturated and partially unsaturated hydrocarbon rings of 3 to 9, preferably 3 to 7 carbon atoms. The term “cycloalkyl” includes such rings having zero to four substituents (preferably 0-2 substituents), selected from the group consisting of halogen, alkyl, substituted alkyl (e.g., trifluoromethyl), alkenyl, substituted alkenyl, alkynyl, nitro, cyano, keto, OR_{d}, SR_{d} NR_{d}Rₑ NR_{c}SO₂, NRₑSO₂R_{c}, C(═O)H, acyl, —CO₂H, alkoxycarbonyl, carbamyl, sulfonyl, sulfonamide, —OC(═O)R_{d}, ═N—OH, ═N—O-alkyl, aryl, heteroaryl, heterocyclo, a 4 to 7 membered carbocyclic ring, and a five or six membered ketal, e.g., 1,3-dioxolane or 1,3-dioxane, wherein R_{c}, R_{d} and Rₑ are defined as above. The term “cycloalkyl” also includes such rings having a phenyl ring fused thereto or having a carbon-carbon bridge of 3 to 4 carbon atoms. Additionally, when a cycloalkyl is substituted with a further ring, i.e., aryl, arylalkyl, heteroaryl, heteroarylalkyl, heterocyclo, heterocycloalkyl, cycloalkylalkyl, or a further cycloalkyl ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “aryl” refers to phenyl, biphenyl, 1-naphthyl, 2-naphthyl, and anthracenyl, with phenyl being preferred. The term “aryl” includes such rings having zero to four substituents (preferably 0-2 substituents), selected from the group consisting of halo, alkyl, substituted alkyl (e.g., trifluoromethyl), alkenyl, substituted alkenyl, alkynyl, nitro, cyano, OR_{d}, SR_{d}, NR_{d}Rₑ, NR_{d}SO₂, NR_{d}SO₂R_{c}, C(═O)H, acyl, —CO₂H, alkoxycarbonyl, carbamyl, sulfonyl, sulfonamide, —OC(═O)R_{d}, heteroaryl, heterocyclo, cycloalkyl, phenyl, benzyl, napthyl, including phenylethyl, phenyloxy, and phenylthio, wherein R_{c}, R_{d} and Rₑ are defined as above. Additionally, two substituents attached to an aryl, particularly a phenyl group, may join to form a further ring such as a fused or spiro-ring, e.g., cyclopentyl or cyclohexyl or fused heterocycle or heteroaryl. When an aryl is substituted with a further ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “heterocyclo” refers to substituted and unsubstituted non-aromatic 3 to 7 membered monocyclic groups, 7 to 11 membered bicyclic groups, and 10 to 15 membered tricyclic groups, in which at least one of the rings has at least one heteroatom (O, S or N). Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The fused rings completing bicyclic and tricyclic groups may contain only carbon atoms and may be saturated, partially saturated, or unsaturated. The nitrogen and sulfur atoms may optionally be oxidized and the nitrogen atoms may optionally be quaternized. The heterocyclo group may be attached at any available nitrogen or carbon atom. The heterocyclo ring may contain zero to four substituents (preferably 0-2 substituents), selected from the group consisting of halo, alkyl, substituted alkyl (e.g., trifluoromethyl), alkenyl, substituted alkenyl, alkynyl, nitro, cyano, keto, OR_{d}, SR_{d}, NR_{d}R_{c}, NR_{d}SO₂, NR_{d}SO₂R_{c}, SO₂R_{d}, C(═O)H, acyl, —CO₂H, alkoxycarbonyl, carbamyl, sulfonyl, sulfonamide, —OC(═O)R_{d}, ═N—OH, ═N—O-alkyl, aryl, heteroaryl, cycloalkyl, a five or six membered ketal, e.g., 1,3-dioxolane or 1,3-dioxane, or a monocyclic 4 to 7 membered non-aromatic ring having one to four heteroatoms, wherein R_{c}, R_{d} and Rₑ are defined as above. The term “heterocyclo” also includes such rings having a phenyl ring fused thereto or having a carbon-carbon bridge of 3 to 4 carbon atoms. Additionally, when a heterocyclo is substituted with a further ring, i.e., aryl, arylalkyl, heteroaryl, heteroarylalkyl, cycloalkyl, cycloalkylalkyl, heterocycloalkyl, or a further heterocyclo ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Exemplary monocyclic groups include azetidinyl, pyrrolidinyl, oxetanyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl.

The term “heteroaryl” refers to substituted and unsubstituted aromatic 5 to 7 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero to four substituents (preferably 0-2 substituents), selected from the group consisting of halo, alkyl, substituted alkyl (e.g., trifluoromethyl), alkenyl, substituted alkenyl, alkynyl, nitro, cyano, OR_{d}, SR_{d}, NR_{d}Rₑ, NR_{d}SO₂, NR_{d}SO₂R_{c}, SO₂R_{d}, C(═O)H, acyl, —CO₂H, alkoxycarbonyl, carbamyl, sulfonyl, sulfonamide, —OC(═O)R_{d}, heterocyclo, cycloalkyl, aryl, or a monocyclic 4 to 7 membered aromatic ring having one to four heteroatoms, including phenylethyl, phenyloxy, and phenylthio, wherein R_{c}, R_{d} and Rₑ are defined as above. Additionally, when a heteroaryl is substituted with a further ring, i.e., aryl, arylalkyl, heterocyclo, heterocycloalkyl, cycloalkyl, cycloalkylalkyl, heteroarylalkyl, or a further heteroaryl ring, such ring in turn may be substituted with one to two of C₀₋₄alkyl optionally substituted with halogen, trifluoromethyl, alkenyl, alkynyl, nitro, cyano, keto (═O), OH, O(alkyl), phenyloxy, benzyloxy, SH, S(alkyl), NH₂, NH(alkyl), N(alkyl)₂, NHSO₂, NHSO₂(alkyl),n SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, OC(═O)NH(alkyl), NHC(═O)alkyl, and NHCO₂(alkyl).

Exemplary monocyclic heteroaryl groups include pyrrolyl, pyrazolyl, pyrazolinyl, imidazolyl, oxazolyl, isoxazolyl, thiazolyl

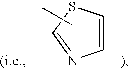

thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

When the term “unsaturated” is used herein to refer to a ring or group, the ring or group may be fully unsaturated or partially unsaturated.

Throughout the specification, groups and substituents thereof may be chosen by one skilled in the field to provide stable moieties and compounds.

The compounds of formula I form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. In addition, when a compound of formula I contains both a basic moiety, such as, but not limited to an amine or a pyridine or imidazole ring, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula I may be formed, for example, by reacting a compound of the formula I with an amount of acid or base, such as an equivalent amount, in a medium such as one in which the salt precipitates or in an aqueous medium followed by lyophilization.

The compounds of formula I which contain a basic moiety, such as, but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The compounds of formula I which contain an acidic moiety, such as, but not limited to a carboxylic acid, may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Compounds of the formula I, and salts thereof, may exist in their tautomeric form (for example, as an amide or imino ether). All such tautomeric forms are contemplated herein as part of the present invention.

All stereoisomers of the present compounds, such as those, for example, which may exist due to asymmetric carbons, including enantiomeric forms (which may exist even in the absence of asymmetric carbons) and diastereomeric forms, are contemplated and within the scope of this invention. Individual stereoisomers of the compounds of this invention may, for example, be substantially free of other isomers, or may be admixed, for example, as racemates or with all other or other selected, stereoisomers. The chiral centers of the present invention can have the S or R configuration as defined by the IUPAC 1974 Recommendations.

In addition, compounds of the formulas I may have prodrug forms. Any compound that will be converted in vivo to provide the bioactive agent (i.e., a compound of formula I) is a prodrug within the scope and spirit of the invention.

For example, pro-drug compounds of the formulas I may be carboxylate ester moieties. A carboxylate ester may be conveniently formed by esterifying any of the carboxylic acid functionalities found on the disclosed ring structure(s).

Various forms of prodrugs are well known in the art. For examples of such prodrug derivatives, see:

Design of Prodrugs, edited by H. Bundgaard, (Elsevier, 1985), and Methods in Enzymology, Vol. 42, p. 309-396, edited by K. Widder, et. al. (Academic Press, 1985);

A Textbook of Drug Design and Development, edited by Krosgaard-Larsen and H. Bundgaard, Chapter 5, “Design and Application of Prodrugs,” by H. Bundgaard, p. 113-191 (1991);

H. Bundgaard, Advanced Drug Delivery Reviews, Vol. 8, p. 1-38 (1992);

H. Bundgaard, et al., Journal of Pharmaceutical Sciences, Vol. 77, p. 285 (1988); and

N. Kakeya, et. al., Chem Phar Bull, Vol. 32, p. 692 (1984).

It should further be understood that solvates (e.g., hydrates) of the compounds of formula I are also with the scope of the present invention. Methods of solvation are generally known in the art.

### Preferred Compounds

Preferred compounds of the present invention are those having the following formula, or salts, hydrates, and prodrugs thereof,

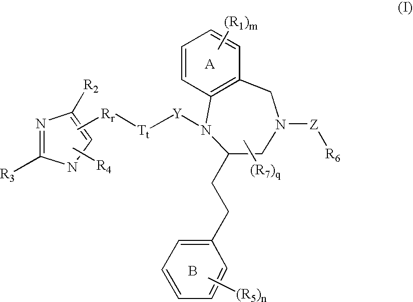

wherein:

R₁ and R₅ are attached to any available carbon atom of phenyl ring A and phenyl ring B, respectively, and at each occurrence are independently selected from alkyl, aralkyl, aminoalkyl, halogen, cyano, nitro, hydroxy, alkoxy, alkylthio, NH₂, NH(alkyl), N(alkyl)₂, C(═O)H, acyl, CO₂H, alkoxycarbonyl, carbamyl, sulfonyl, sulfonamide, cycloalkyl, heterocycle, aryl, and heteroaryl, and/or two of R₁ and/or two of R₅ join together to form a fused benzo ring;

R₂, R₃ and R₄ are independently selected from hydrogen and alkyl;

Z is —CO₂—, —SO₂—, or is absent;

Y, R and T are selected from —CH₂— and —C(═O)—,

R₆ is selected from:

C₁₋₄alkyl or C₁₋₄alkenyl optionally substituted with up to three of halogen, aryl and CO₂C₁₋₆alkyl;

phenyl optionally substituted with up to three R₁₂ and/or having fused thereto a benzo-ring or a five to six membered heteroaryl;

heteroaryl selected from thiophenyl, imidazolyl, pyrazolyl, and isoxazolyl wherein said heteroaryl is optionally substituted with up to two R₁₂,

provided that where R₂ is hydrogen, Z-R₆ together are not —SO₂-Me or

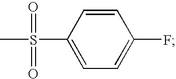

R₇ is selected from hydrogen, alkyl, substituted alkyl, alkenyl, substituted alkenyl, aminoalkyl, halogen, cyano, nitro, keto (═O), hydroxy, alkoxy, alkylthio, C(═O)H, acyl, CO₂H, alkoxycarbonyl, carbamyl, sulfonyl, sulfonamide, cycloalkyl, heterocycle, aryl, and heteroaryl;

R₁₂ at each occurrence is independently selected from each other R₁₂ from the group consisting of C₁₋₆alkyl, halogen, nitro, cyano, hydroxy, alkoxy, NHC(═O)alkyl, —CO₂alkyl, —SO₂phenyl, five to six membered monocyclic heteroaryl, and phenyloxy or benzyloxy in turn optionally substituted with halogen, C₁₋₄alkyl, and/or O(C₁₋₄alkyl);

m and n are independently selected from 0, 1, 2 or 3; and

q is 0, 1 or 2; and

r and t are 0 or 1.

More preferred are compounds having the following formula, or salts, hydrates, or prodrugs thereof,

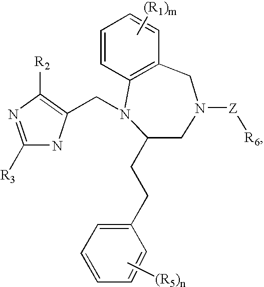

wherein

R₁ and R₅ are attached to any available carbon atom of phenyl ring A and phenyl ring B, respectively, and at each occurrence are independently selected from alkyl, halogen, cyano, hydroxy, alkoxy, NH₂, NH(alkyl), N(alkyl)₂, C(═O)H, acyl, CO₂H, alkoxycarbonyl, and/or two of R₁ and/or two of R₅ join together to form a fused benzo ring;

R₂, R₃ and R₄ are independently selected from hydrogen and lower alkyl;

Z is —CO₂—, —SO₂—, or is absent;

R₆ is selected from:

C₁₋₄alkyl or C₁₋₄alkenyl optionally substituted with up to three of halogen, aryl and CO₂C₁₋₆alkyl;

phenyl optionally substituted with up to three R₁₂ and/or having fused thereto a benzo-ring or a five to six membered heteroaryl;

heteroaryl selected from thiophenyl, imidazolyl, pyrazolyl, and isoxazolyl, wherein said heteroaryl is optionally substituted with up to two R₁₂,

provided that where R₂ is hydrogen, Z-R₆ together are not —SO₂-Me or

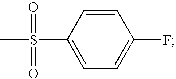

R₁₂ at each occurrence is independently selected from each other R₁₂ from the group consisting of C₁₋₆alkyl, halogen, nitro, cyano, hydroxy, alkoxy, NHC(═O)alkyl, —CO₂alkyl, —SO₂phenyl, five to six membered monocyclic heteroaryl, and phenyloxy or benzyloxy in turn optionally substituted with halogen, C₁₋₄alkyl, and/or O(C₁₋₄alkyl); and

m and n are independently selected from 0, 1, or 2.

Even more preferred are compounds as immediately defined above wherein R₆ is selected from C₁₋₄alkyl, trifluoromethyl, benzyl, C₂₋₃alkenyl substituted with phenyl,

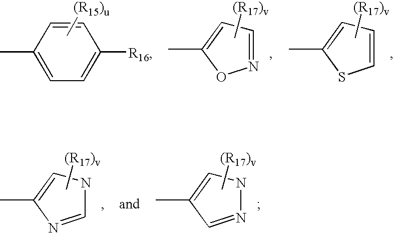

wherein:

R₁₅ is halogen, alkyl, nitro, cyano, hydroxy, alkoxy, NHC(═O)alkyl, and/or two R₁₅ groups are taken together to form a fused benzo ring or a five to six membered heteroaryl;

R₁₆ is selected from hydrogen, halogen, alkyl, nitro, cyano, hydroxy, alkoxy, NHC(═O)alkyl, and phenyloxy or benzyloxy in turn optionally substituted with 1 to 3 of halogen, cyano, and C₁₋₄alkoxy;

R₁₇ is selected from alkyl, alkoxy, CO₂C₁₋₆alkyl, and SO₂phenyl; and

u and v are independently 0, 1 or 2.

Most preferred are compounds having the formula:

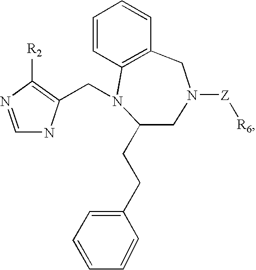

wherein

R₂ is hydrogen or CH₃;

Z is —CO₂—, —SO₂—, or is absent; and

R₆ is selected from the groups recited immediately above, most preferably

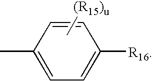

### Utility

The compounds of this invention by inhibiting F₁F₀-ATPase may be used to help conserve ATP under conditions of oxygen deprivation. Thus, the compounds may be useful in treating or preventing any condition associated with depleted levels of ATP and/or tissue ischemia (from mild to acute or severe). As used herein with reference to the utilities described below, the terms “treating” or “treatment” encompass both responsive and prophylaxis measures designed to inhibit or delay the onset of the disease or disorder, or to alleviate, ameliorate, lessen, or cure the disease or disorder and/or its symptoms.

In view of their F₁F₀-ATPase inhibitory activity, the inventive compounds are useful in treating cardiovascular diseases including, without limitation, congestive heart failure, cardiac arrhythmias, unstable angina, and high blood pressure. The compounds also are useful to treat ischemia, including ischemia resulting from vascular occlusion, cerebral infarction, stroke and related cerebral vascular diseases (including cerebrovascular accident and transient ischemic attack), and accurate coronary syndromes such as myocardial infarction, coronary artery disease, unstable angina, and non-Q wave MI.

Additionally, the compounds are useful in treating or preventing symptoms or consequences occurring from thrombosis and/or the formation of atherosclerotic plaques, atherosclerosis, peripheral arterial disease, coagulation syndromes, and intermittent claudication. The compounds may be used to treat thrombotic or thromboembolic conditions such as thromboembolic stroke (including that resulting from atrial fibrillation or from ventricular mural thrombus); venous thrombosis (including deep vein thrombosis); arterial thrombosis; cerebral thrombosis; pulmonary embolism; cerebral embolism; peripheral occlusive arterial disease (e.g., peripheral arterial disease, intermittent claudication, critical leg ischemia, prevention of amputation, prevention of cardiovascular morbidity such as MI, stroke or death); thromboembolic consequenses of surgery, interventional cardiology or immobility; thromboembolic consequenses of medication (such as oral contraceptives, hormome replacement and heparin); thrombotic consequenses of atherosclerotic vascular disease and atherosclerotic plaque rupture leading to tissue ischemia; prevention of atherosclerotic plaque formation; transplant atherosclerosis; thromboembolic complications of pregancy including fetal loss; thromboembolic consequences of thrombophilia (e.g., Factor V Leiden, and homocystinenimia); prothrombotic consequences and/or complications of cancer; prevention of thrombosis on artificial surfaces (such as stents, blood oxygenators, shunts, vascular access ports, vascular grafts, artificial valves, etc.); coagulopathies (e.g., disseminated intravascular coagulation); coagulation syndromes; vascular remodeling atherosclerosis, restenosis and systemic infection; prevention of metastesis and tumor implantation; diabetic complications including retinopathy, nephropathy and neuropathy; inflammation; Kasabach-Merritt syndrome; atrial fibrillation; ventricular enlargement (including dilated cardiac myopathy and heart failure); restenosis (e.g., following arterial injury-induced either endogenously or exogenously).

Compounds of the present invention may be useful for maintaining blood vessel patency in conjunction with vascular surgery including bypass grafting, arterial reconstruction, atherectomy, vascular graft and stent patency, organ, tissue and cell implantation and transplantation. In addition, the compounds of the present invention may be useful for maintaining blood vessel patency in conjunction with interventional cardiology or vascular surgery including bypass grafting, arterial reconstruction, atherectomy, vascular graft and stent patency, organ, tissue and cell implantation and transplantation. Additionally, the compounds may be used for preservation of tissue as related to organ transplantation.

The inventive compounds also are useful in treating diseases or disorders in other tissues or muscles that are associated with ischemic conditions. For example, the compounds may be used to treat muscle cell damage and necrosis.

Additionally, the inventive compounds may be useful as anti-cancer and/or anti-tumor agents. It is reported that inhibitors of mitochondrial F₁F₀-ATPase selectively kill metabolically active tumor cells that do not exhibit the Warburg effect, i.e., cells that do not maintain a high level of anaerobic carbon metabolism even in the presence of oxygen. See Salomon et al., “Understanding and Exploiting the Mechanistic Basis for Selecivity of Polyketide Inhibitors of F₁F₀-ATPase,” Proc. Natl. Acad. Sci. Vol. 97 (26) (2000), at pp.14766-14771. Accordingly, the compounds of the present invention are useful in treating tumor growth, as an adjunct to chemotherapy, and for treating cancer, more particularly, cancer of the lung, prostate, colon, breast, ovaries, and bone.

The inventive compounds may also be used in combination with other F₁F₀-ATPase inhibitors such as efrapeptin, oligomycin, autovertin B, and azide, and/or in combination with other cardiovascular drugs. Additionally, the compounds may be used in combination with other therapeutic agents such as potassium channel openers, calcium channel blockers, sodium hydrogen exchanger inhibitors, anti-arrhythmic agents, thrombin inhibitors, platelet aggregation inhibitors or anti-platelet agents, fibrinogen antatagonists, diuretics, anti-hypertensive agents, mineralocorticoid receptor antagonists; phospodiesterase inhibitors; cholesterol/lipid lowering agents and lipid profile therapies; anti-diabetic agents; anti-depressants; anti-inflammatory agents (steroidal and non-steroidal); anti-oxidant agents; angiogenesis modulators; anti-osteoporosis agents; hormone replacement therapies; oral contraceptives; anti-coagulants; anti-obesity agents; anti-anxiety agents; anti-proliferative agents; anti-tumor agents; anti-ulcer and gastroesophageal reflux disease agents; growth hormone and/or growth hormone secretagogues; thyroid mimetics (including thyroid receptor antagonist); anti-infective agents; anti-viral agents; anti-bacterial agents; and anti-fungal agents.

For example, the inventive compounds may be used in combination with aspirin, clopidogrel, ticlopidine or CS-747, warfarin, and low molecular weight heparins (such as lovenox, enoxaparain, and dalteparin). Other suitable therapeutic agents in combination with which the inventive compounds may be used include:

anti-arrhythmic agents including Class I agents (such as propafenone); Class II agents (propranolol); Class III agents (such as sotalol, dofetilide, amiodarone, azimilide and ibutilide); Class IV agents (such as ditiazem and verapamil); K⁺ channel openers such as I_{Ach} inhibitors, and I_{Kur} inhibitors (e.g., compounds such as those disclosed in U.S. application Ser. No. 09/729,731, filed Dec. 5, 2000;

alpha- or beta- adrenergic blockers (such as propranolol, nadolol and carvedilol), or -β-adrenergic agonists such as albuterol, terbutaline, formoterol, salmeterol, bitolterol, pilbuterol, and/or fenoterol;

angiotensin-II receptor antagonists (e.g., irbesartan, losartan or valsartan);

anticholinergics such as ipratropium bromide;

anti-diabetic agents such as biguanides (e.g. metformin); glucosidase inhibitors (e.g. acarbose); insulins (including insulin secretagogues or insulin sensitizers); meglitinides (e.g. repaglinide); sulfonylureas (e.g., glimepiride, glyburide and glipizide); biguanide/glyburide combinations (e.g., glucovance), thiozolidinediones (e.g. troglitazone, rosiglitazone and pioglitazone), PPAR-alpha agonists, PPAR-gamma agonists, PPAR alpha/gamma dual agonists, SGLT2 inhibitors, inhibitors of fatty acid binding protein (aP2) such as those disclosed in U.S. Ser. No. 09/519,079 filed Mar. 6, 2000 and assigned to the present assignee, glucagon-like peptide-1 (GLP-1), and dipeptidyl peptidase IV (DP4) inhibitors;

anti-depressant or anti-anxiety agents such as nefazodone, sertraline, diazepam, lorazepam, buspirone, and hydroxyzine pamoate;

anti-diabetic agents such as biguanides (e.g. metformin); glucosidase inhibitors (e.g. acarbose); insulins (including insulin secretagogues or insulin sensitizers); meglitinides (e.g. repaglinide); sulfonylureas (e.g., glimepiride, glyburide and glipizide); biguanide/glyburide combinations (e.g., glucovance), thiozolidinediones (e.g. troglitazone, rosiglitazone and pioglitazone), PPAR-alpha agonists, PPAR-gamma agonists, PPAR alpha/gamma dual agonists, SGLT2 inhibitors, inhibitors of fatty acid binding protein (aP2) such as those disclosed in U.S. Ser. No. 09/519,079 filed Mar. 6, 2000 and assigned to the present assignee, glucagon-like peptide-1 (GLP-1), and dipeptidyl peptidase IV (DP4) inhibitors;

anti-hypertensive agents such as angiotensin-converting enzyme (ACE) inhibitors (e.g., captopril, lisinopril, zofenopril, ramipril, fosinopril, enalapril, ceranopril, cilazopril, delapril, pentopril, quinapril), vasopeptidase inhibitors, i.e., dual ACE/NEP inhibitors (e.g., omapatrilat and gemopatrilat), AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/AII antagonist (e.g., compounds disclosed in WO 00/01389); neutral endopeptidase (NEP) inhibitors;

anti-inflammatory agents such as cromolyn, nedocromil, theophylline, zileuton, zafirlukast, monteleukast and/or pranleukast or cortiocosteroids including beclomethasone, triamcinolone, budesonide, fluticasone, flunisolide or dexamethasone; prednisone; dexamethasone; enbrel; protien tyrosine kinase (PTK) inhibitors; cyclooxygenase inhibitors (including NSAIDs, and COX-1 and/or COX-2 inhibitors); aspirin; or indomethacin; lipoxygenase inhibitors; chemokine receptor modulators (including CCR_{1,} CCR_{2,} CCR_{3,} CXCR₂ receptor antagonists); secretory and cytosolic phospholipase A2 inhibitors; VLA4 antagonists; cytokine modulators (e.g. TNF-alpha converting enzyme (TACE) inhibitors, Interleukin-1 converting enzyme (ICE) inhibitors, Interleukin-1 receptor antagonists);

angiogenesis modulators such as endostatin;

anti-oxidant agents and/or lipid peroxidation inhibitors such as probucol, BO-653, Vitamin A, Vitamin E, AGI-1067;

anti-platelet agents such as GPIIb/GPIIIa blockers, (e.g., abciximab, eptifibatide, tirofiban); P2Y₁₂ antagonists (e.g., clopidogrel, ticlopidine, CS-747); or thromboxane receptor antagonists (e.g., ifetroban);

anti-osteoporosis agents including alendronate and raloxifene.

anti-obesity agents including orlistat and aP2 inhibitors (such as those disclosed in U.S. Ser. No. 09/519,079 filed Mar. 6, 2000);

anti-proliferative agents for use in combination with the compounds of the present invention include cyclosporin A, paclitaxel, FK 506, and adriamycin;

anti-ulcer and gastroesophageal reflux disease agents including famotidine, ranitidine, and omeprazole;

sodium hydrogen exchanger-1 (NHE-1) inhibitors such as cariporide;

calcium channel blocking agents such as verapamil, nifedipine, diltiazem, amlodipine and mybefradil;

cardiac glycosides such as digitalis and ouabain;

diuretics such as chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride;

hormone replacement therapies including estrogen (e.g., congugated estrogens) and estradiol;

lipid profile modulators including HMG-CoA reductase inhibitors (e.g., pravastatin, simvastatin, atorvastatin, fluvastatin, cerivastatin, AZ4522, itavastatin [Nissan/Kowa]), ZD-4522 (a.k.a. rosuvastatin, or atavastatin or visastatin)); squalene synthetase inhibitors; fibrates; bile acid sequestrants (such as questran); ACAT1 inhibitors; ACAT2 inhibitors; dual ACAT1/2 inhibitors; MTP inhibitors; cholesterol absorption inhibitors; and cholesterol ester transfer protein inhibitors (e.g., CP-529414); PPAR-delta agonists; PPAR-alpha agonists; dual PPAR-alpha/delta agonists; LXR-alpha agonists; LXR-beta agonists; LXR dual alphalbeta agonists;

mineralocorticoid receptor antagonists such as spironolactone and eplirinone.

microsomal triglyceride transport protein inhibitors (such as disclosed in U.S. Pat. Nos. 5,739,135, 5,712,279 and 5,760,246);

phosphodiesterase (PDE) inhibitors including dipyridamole, cilostazol, or sildenafil, or PDE inhibitors in combination with aspirin, ifetroban, picotamide, ketanserin, clopidogrel, and/or thromboxane receptor antagonists or thromboxane A synthetase inhibitors (such as picotamide);

serotonin-2-receptor antagonists (such as ketanserin), fibrinogen receptor antagonists, and

thrombolytic agents, such as tissue plasminogen activator (natural or recombinant), streptokinase, reteplase, activase, lanoteplase, urokinase, prourokinase, tenecteplase (TNK), lanoteplase (nPA), anisolated streptokinase plasminogen activator complex (ASPAC), factor VIIa inhibitors, factor Xa inhibitors, thrombin inhibitors (such as hirudin and argatroban), animal salivary gland plasminogen activators, PAI-1 inhibitors such as XR-330 and T-686, and inhibitors of α-2-antiplasmin such as anti-α-2-antiplasmin antibody, prostacyclin mimetics.

The inventive compounds may also be useful in combination with other anticancer strategies and chemotherapies such as taxol and/or cisplatin. The compounds may be used in conjunction with anti-tumor agents such as paclitaxel, adriamycin, epithilones, cisplatin, and carboplatin.

The various other therapeutic agents described above may be employed in the same dosage form with the compound of formula I or in different dosage forms, in dosages and regimens as generally known in the art or in the PDR.

The compounds of the present invention may act in a synergistic fashion with one or more of the above agents to allow for increased efficacy and/or reduced doses of any of the above agents and therefore minimize potential hemorrhagic side-effects.

The compounds of formula I may be administered by any means suitable for the condition to be treated. Systematic treatment is typically preferred for cancerous conditions, although other modes of delivery are contemplated. The compounds may be delivered orally, such as in the form of tablets, capsules, granules, powders, or liquid formulations including syrups; sublingually; bucally; transdermally; parenterally, such as by subcutaneous, intravenous, intramuscular or intrastemal injection or infusion (e.g., as sterile injectable aqueous or non-aqueous solutions or suspensions); nasally such as by inhalation spray; rectally such as in the form of suppositories; or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The effective amount of a compound of the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. An exemplary effective amount of compounds of formula I may be within the dosage range of about 0.1 to about 100 mg/kg, preferably about 0.2 to about 50 mg/kg and more preferably about 0.5 to about 25 mg/kg (or from about 1 to about 2500 mg, preferably from about 5 to about 2000 mg) on a regimen in single or 2 to 4 divided daily doses.

### Assay

Mitochondria were isolated from bovine hearts and purified through a Percoll gradient, sonicated to generate sub mitochondrial particles (SMP), centrifuged, and stored at −80° C. See Gasnier F. et al, “Use of Percoll Gradients for Isolation of Human Placenta Mitochondria Suitable for Investigating Outer Membrane Proteins,” Anal. Biochem., Vol 212(1) (1993) at pp. 173-178; and Matsuno-Yagi A et al, “Studies on the Mechanism of Oxidative Phosphorylation: Effects of Specific F₀ Modifiers on Ligand-Induced Conformation Changes of F₁,” Proc. Nat'l Acad. Sci. USA, Vol. 82(22) (1985), at pp. 7550-7554.) ATP hydrolyase activity was determined using SMP and the well-characterized coupled enzyme system in which ATP hydrolysis and subsequent ADP generation is coupled through pyruvate kinase and lactate dehydrogenase to NAD+ generation which was monitored by a decrease in absorbance at 340 nm (see Pullman, M. E. et al, “Partial Resolution of the Enzymes Catalyzing Oxidative Phosphorylation,” J. Biol. Chem. Vol. 235 (1960), at pp. 3322-3329.) Similarly, compound effects on ATP synthase activity were determined using SMP in the well-characterized coupled enzyme assay in which ATP generation is coupled to NADPH synthesis through the hexokinase and glucose-6-phosphate dehydrogenase pathway (Cross & Kohlbrenner, “The Mode of Inhibition of Oxidative Phosphorylation by Efrapeptin (A23871). Evidence for an Alternating Site Mechanism for ATP Synthesis,” J. Biol. Chem., Vol. 253 (1978) at pp. 4865-4873.) NADPH increase was monitored spectrophotometrically by an increase in absorbance at 340 nm. Compounds were dissolved in 100% dimethyl sulfoxide and tested at increasing concentrations for enzyme inhibition. The concentration of compound causing 50% inhibition of the enzyme (IC₅₀) was calculated after the data was fitted using the Levenburg Marquardt algorithm and Microsoft Excel.

Compounds of formula (I), and more particularly, the compounds of Examples 1 through 39 hereof, were tested in this assay and found to have activity for inhibiting F₁F₀-ATP hydrolase.

### Abbreviations

The following abbreviations are employed in the Examples and elsewhere herein:

Ph=phenyl

Bn=benzyl

Me=methyl

Et=ethyl

Pr=propyl

Bu=butyl

MeOH=methanol

AcOH=acetic acid

DBU=1,8-diazabicyclo[5,4,0]undec-7-ene

DCE=1,2-dichloroethane

DIP-Cl=B-chlorodiisopinocampheylborane

DMF=N,N-dimethylformamiide

DPPA=Diphenylphosphoryl azide

EDC=N-(3-dimethylaminopropyl)-N′-ethylcarbodiimide

EtOAc=ethyl acetate

NaBH₄=sodium borohydride

NaHCO₃=sodium bicarbonate

KCNS=potassium isothiocyanate

Pd/C=palladium on carbon

PtO₂=platinum oxide

Ph₃P=triphenylphosphine

TEA=triethylamine or Et₃N

THF=tetrahydrofuran

TFA=trifluoroacetic acid

min=minute(s)

h or hr=hour(s)

L=liter

mL=milliliter

μL=microliter

g=gram(s)

mg=milligram(s)

mol=mole(s)

mmol=millimole(s)

meq=mnilliequivalent

rt=room temperature

sat or sat'd=saturated

aq.=aqueous

TLC=thin layer chromatography

LC/MS=high performance liquid chromatography/mass spectrometry

MS or Mass Spec=mass spectrometry

mp=melting point

### Process of Preparation

Inventive compounds that are inhibitors of mitochondrial F₁F₀ ATP hydrolase may be prepared by methods illustrated in Schemes I through III below. Starting materials are commercially available or can be readily prepared by one of ordinary skill in the art using known methods. For all of the schemes and compounds, the groups R₁-R₄ are as described above for a compound of Formula I.

Solvents, temperatures, pressures, and other reaction conditions may readily be selected by one of ordinary skill in the art. High Speed Analoging (HSA) may be used in preparing compounds, for example, where the intermediates possess a carboxylic acid or amino group. For ease of reference, abbreviations listed above are used in these schemes.

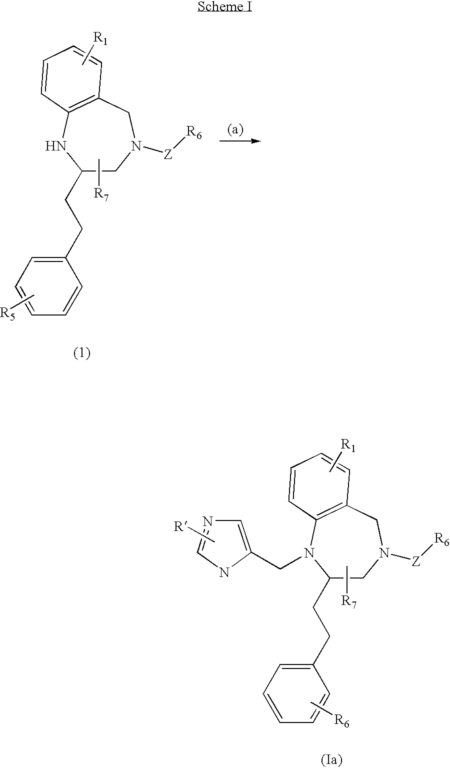

imidazolylaldehyde, Na(OAc)₃BH, DCE, HOAc.

The tetrahydrobenzodiazepine (1) was reductively alkylated with a heteroaryl aldehyde such as 4-formylimidazole using a reducing agent like sodium triacetoxyborohydride in a solvent like dichloroethane/AcOH, to provide the compounds of formula (Ia) as shown in Scheme I.

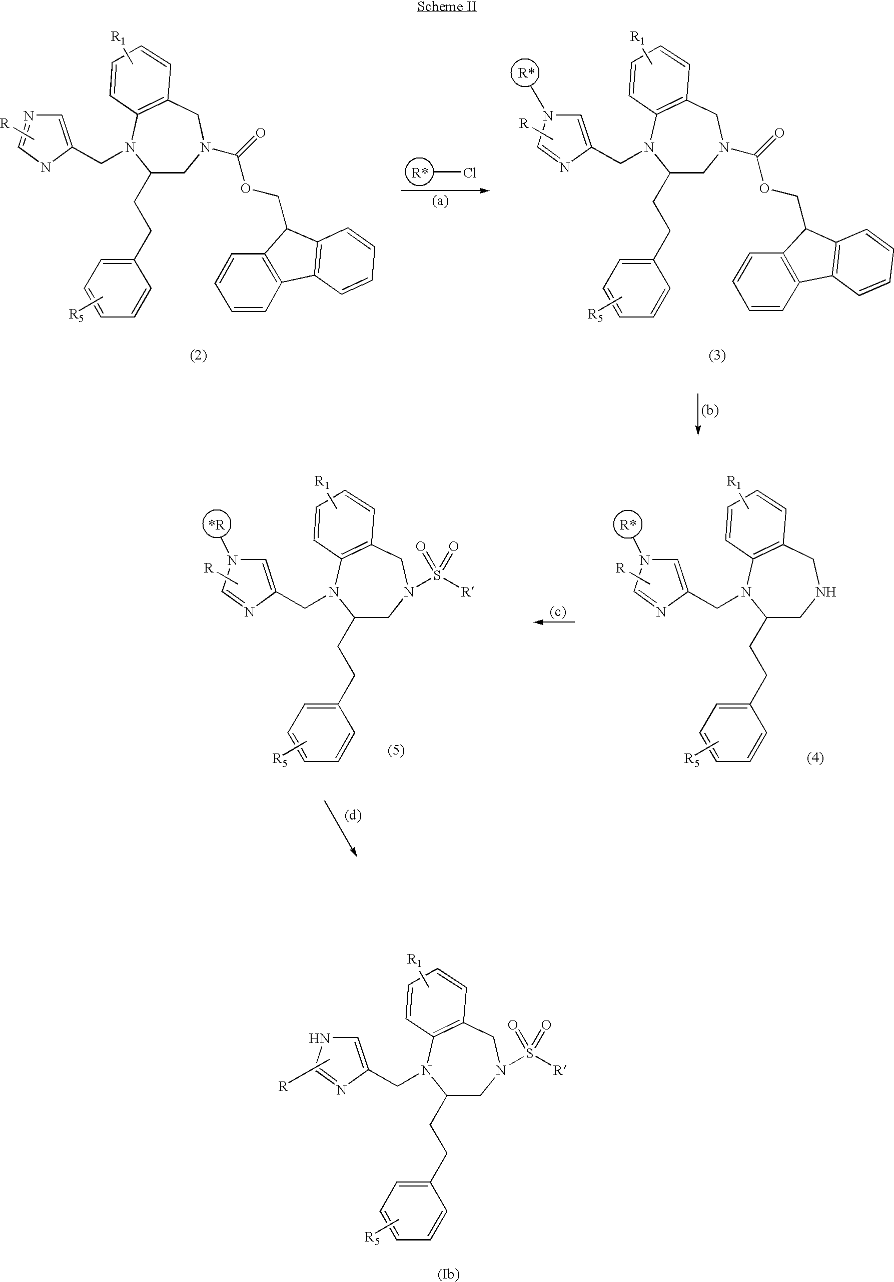

2-Chlorotrityl chloride, DIPEA, DMF; (b) piperidine, DMF; (c) R′SO₂Cl, DIPEA; (d) TFA, CH₂Cl₂.

A high through-put synthesis was used to produce compounds of formula (Ib) as described in Scheme II. The FMOC-protected imidazolylbenzodiazepine (2) was attached to readily cleavable polymer such as 2-chlorotrityl resin (designated

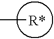

), to produce compounds (3). The FMOC group was removed by treatment with an amine base such as piperidine to produce compounds (4). The exposed benzodiazepine amine (4) was reacted with a sulfonyl chloride to produce compounds (5), followed by cleavage from the polymer to give compounds of formula (Ib).

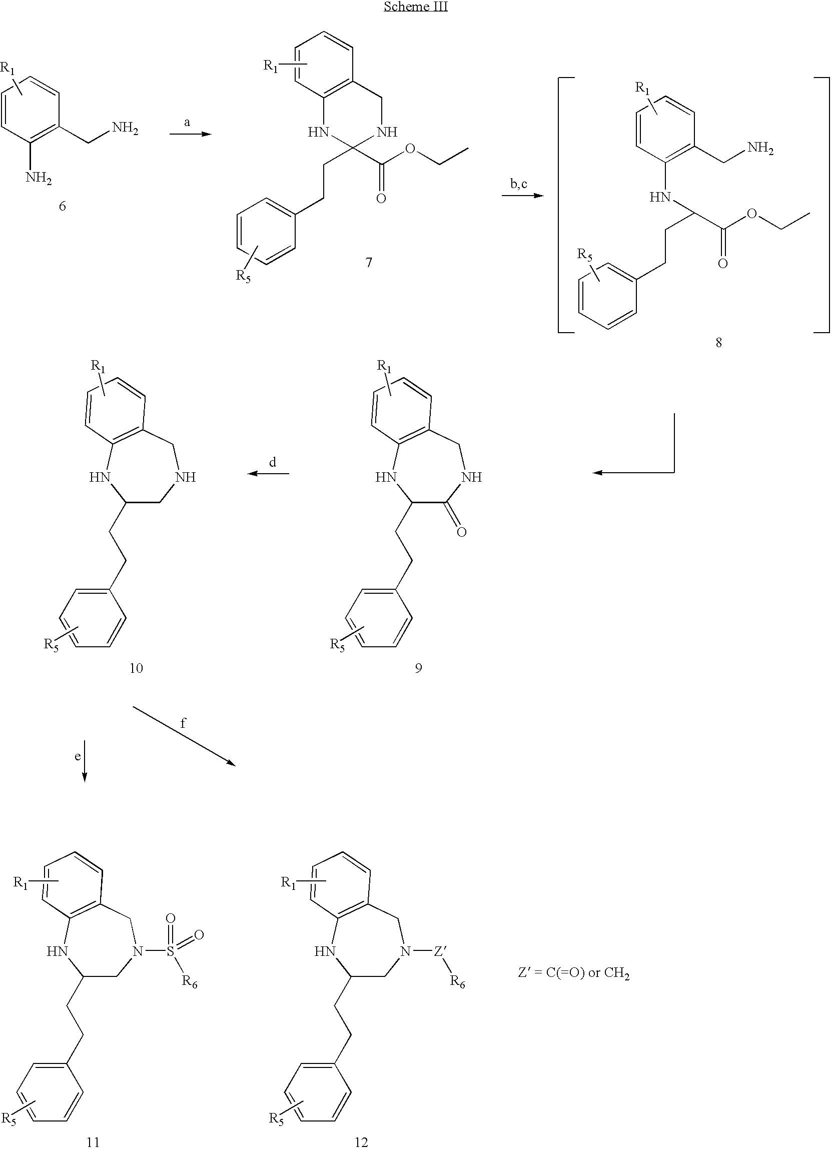

Ph(CH₂)₂C(═O)CO₂Et, toluene, heat; (b) DCE, TFA, Et₃SiH; (c) NaOH/MeOH; (d) LAH, THF; (e) RS(═O)₂Cl, CH₂Cl₂; (f) NaH, DMF, Z-Br.

The precursors to the compounds of formula (I) were prepared as shown in Scheme III. 2-Aminobenzylamine (6) was reacted with an α-ketoester in refluxing toluene to give tetrahydroquinazoline (7). Reduction of (7) with triethylsilane in TFA and 1,2-dichloroethane gave tetrahydrobenzodiazepin-3-one (9) through the intermediacy of amino ester (8). Reduction of (9) to give diamine (10) was achieved with lithium aluminum hydride in THF. The diamine (10) could then be sulfonylated with a sulfonyl chloride to give precursors (11) of formula I, or acylated or alkylated under standard conditions to give N-acyl- or N-alkyl-substituted tetrahydrobenzodiazepines (12).

The invention will now be further described by the following working examples, which are preferred embodiments of the invention. All temperatures are in degrees Celsius (° C.) unless otherwise indicated. These examples are illustrative rather than limiting.

### EXAMPLE 1

### 4-(4-Fluorophenylsulfonyl)-1-(4-methylimidazol-5-ylmethyl)-2-phenylethyl-2,3,4,5-tetrahydrobenzodiazepine

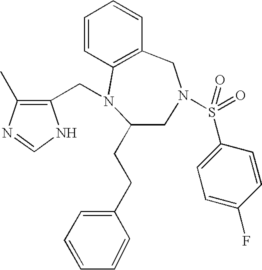

2-Ethoxycarbonyl-2-phenylethyl-1,2,3,4-tetrahydroquinazoline

A solution of 2-aminobenzylamine (2.4 g, 20 mmol), and ethyl 2-oxo-4-phenylbutanoate in toluene was heated at reflux using a Dean-Stark apparatus to remove water for 18 h. The solvent was removed to give Compound A as a brown oil (6 g, 95%). ¹H NMR (CDCl₃) δ1.20 (m, 3H), 2.10 (m, 2H), 2.33 (m, 1H), 2.40 (m, 1H), 4.00 (dd, 2H), 4.70 (m, 2H), 6.60 (d, 1H), 6.75 (t, 1H), 6.90 (m, 1H), 7.05 (t, 1H), 7.15-7.35 (m, 5H).

2-Phenylethyl-2,3,4,5-tetrahydrobenzodiazepin-3-one

To a stirred solution of Compound A (4.5 g, 14.5 mmol) in DCE (40 mL) was added TFA (10 mL) at 0° C., followed by triethylsilane (4.0 mL, 25 mmol). The stirred mixture was slowly allowed to warm to rt over 2 h. The solvent was removed and the residue was dissolved in MeOH (50 mL). To this stirred solution at 0° C. was added 1 N NaOH to adjust the pH to 13, and stirring was continued for 18 h. The precipitate was collected to give Compound B as a tan solid (2.5 g, 65%). ¹H NMR (CDCl₃) δ1.95 (m, 1H), 2.30 (m, 1H), 2.79 (m, 2H), 3.55 (d, J=5 Hz, 1H), 3.86 (dd, J=7, 16 Hz, 1H), 4.32(dd, J=6.5, 12 Hz, 1H), 4.86 (dd, J=7, 16 Hz, 1H), 6.50 (d, J=8 Hz, 1H), 6.65 (m, 2H), 6.90 (d, J=7.4 Hz, 1H), 7.07 (m, 1H), 7.20 (5H). ¹³C NMR (CDCl₃) δ31.34, 31.71, 44.49, 52.94, 116.76, 117.51, 120.45, 125.28, 127.66, 127.68, 127.93, 128.35, 140.32, 144.54.

Phenylethyl-2,3,4,5-tetrahydrobenzodiazepine

To a stirred suspension of lithium aluminum hydride in THF at rt under argon was added a solution of Compound B (2.3 g, 8.6 mmol) in THF through an addition funnel. The mixture was allowed to stir at rt for 18 h, then the reaction was quenched by addition of water (1 mL). The resultant suspension was filtered, and the filtrate was concentrated to give the Compound C as a yellow oil (2.1 g, 98%), which was used directly in the next step. MS: 253 (M+H).

4-(4-Fluorophenylsulfonyl)-2-phenylethyl-2,3,4,5-tetrahydrobenzodiazepine

To a stirred solution of Compound C (280 mg, 1.1 mmol) in CH₂Cl₂ was added TEA and 4-fluorobenzenesulfonyl chloride (220 mg) sequentially. The mixture was allowed to stir at rt for 3 h, and then saturated potassium carbonate solution was added, followed by solid potassium carbonate. The suspension was filtered, and the filtrate was concentrated in vacuo to give the Compound D as a yellow oil (300 mg, 68%). ¹H NMR (CDCl₃) δ1.79 (m, 2H), 2.72 (m, 2H), 3.00 (m, 1H), 3.30 (dd, 1H), 3.58 (dd, 1H), 4.28 (d, J=15 Hz, 1H), 4.45 (d, J=15 Hz, 1H), 6.58 (d, J=8.0 Hz, 1H), 6.80-7.40 (m, 10H), 7.60 (m, 2H). ¹³C NMR (CDCl₃) δ32.32, 34.68, 52.39, 55.61, 115.73, 116.06, 119.70, 121.20, 126.24, 127.15, 128.27, 128.61, 129.69, 129.81, 135.79, 140.65, 147.11.

4-(4-Fluorophenylsulfonyl)-1-(4-methylimidazol-5-ylmethyl)-2-phenylethyl-2,3,4,5-tetrahydrobenzodiazepine.

To a stirred solution of Compound D (80 mg, 0.2 mmol) in DCE and acetic acid was added 4-methylimidazolealdehyde, and the mixture was allowed to stir at rt for 30 min. Sodium triacetoxyborohydride was then added, and additional quantities of the reagents were added to ensure completion of the reaction. The reaction mixture was then poured into EtOAc and ammonium hydroxide solution, and the organic layer was separated, dried, and concentrated to give the title compound (Example 1) as a solid (95 mg, 90%). ¹H NMR (CDCl₃) δ1.43 (m, 2H), 2.03 (m, 3H), 2.44 (m, 1H), 2.63 (m, 1 H), 3.13 (m, 1H), 3.71 (m, 2H), 4.12 (m, 3H), 4.60 (d, J=10.8 Hz, 1H), 6.90-7.40 (m, 12H), 7.81 (m, 2H). MS: 505 (M+H).

### EXAMPLES 2-3

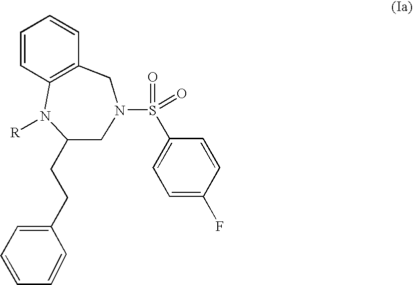

Compounds having the formula (Ia) wherein R has the values listed in Table 1 were prepared by following General Procedure A, as described for the preparation of 4-(4-fluorophenylsulfonyl)-1-(4-methylimidazol-5-ylmethyl)-2-phenylethyl-2,3,4,5-tetrahydrobenzodiazepine (step E of Example 1), except different substituted imidazolealdehydes were used.

TABLE 1

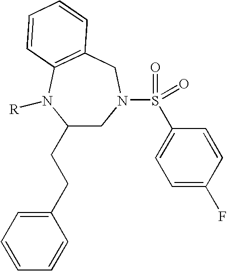

| Example # | Structure (R) | Characterization |
|---|---|---|
| 2 | 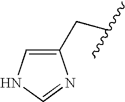 | MS 491 [M + H]⁺ |
| 3 | 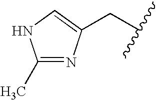 | MS 505 [M + H]⁺ |

### EXAMPLES 4-7

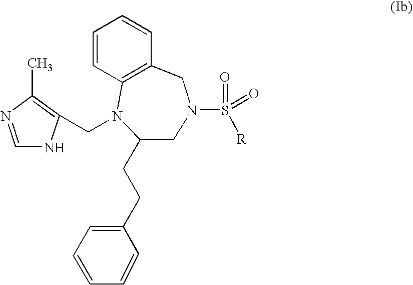

Compounds having the formula (Ib) wherein R has the values listed in Table 2 were prepared according to General Procedure A, as described for the preparation of 4-(4-fluorophenylsulfonyl)-1-(4-methylimidazol-5-ylmethyl)-2-phenylethyl-2,3,4,5-tetrahydrobenzodiazepine (step A to E of Example 1), except different sulfonyl chlorides were used in step D.

TABLE 2

| Example # | Structure (R) | Characterization |
|---|---|---|
| 4 | 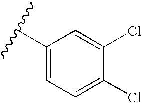 | MS 555 [M + H]⁺ |
| 5 | 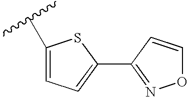 | MS 636 [M + H]⁺ |
| 6 | 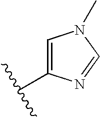 | MS 567 [M + H]⁺ |
| 7 | 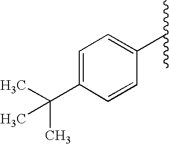 | MS 543 [M + H]⁺ |

### EXAMPLE 8

### Methyl 1-[1-(4-methylimidazol-5-ylmethyl)-2-phenylethyl-2,3,4,5-tetrahydrobenzodiazepin-4-yl]-1-phenylacetate

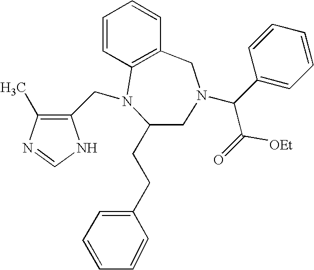

Methyl 2-phenylethyl-2,3,4,5-tetrahydrobenzodiazepin-4-yl-1-phenylacetate.

To a stirred solution of 2-phenylethyl-2,3,4,5-tetrahydrobenzodiazepine (250 mg, 1.0 mmol) in MeOH was added α-bromophenylacetate (200 μL), followed by solid potassium carbonate. The mixture was allowed to stir at rt for 2 h, and then the solvent was removed and the residue partitioned between EtOAc and water. The organic layer was separated, dried, and concentrated, and the residue was purified by silica gel column chromatography (EtOAc/hexanes, 1:4) to give Compound A as an oil (280 mg, 70%). MS: 401 (M+H).

Methyl 1-[1-(4-methylimidazol-5-ylmethyl)-2-phenylethyl-2,3,4,5-tetrahydrobenzodiazepin-4-yl]-1-phenylacetate.

The title compound was prepared by following the same procedure as employed for the preparation of 4-(4-fluorophenylsulfonyl)-1-(4-methylimidazol-5-ylmethyl)-2-phenylethyl-2,3,4,5-tetrahydrobenzodiazepine, except methyl 2-phenylethyl-2,3,4,5-tetrahydrobenzodiazepin-4-yl-1-phenylacetate was used as substrate. The title compound (Example 7) was obtained as a solid. MS: 495 (M+H).

### EXAMPLE 9

### 4-(1,1-Dimethylethoxycarbonyl)-1-(4-methylimidazol-5-ylmethyl)-2-phenylethyl-2,3,4,5-tetrahydrobenzodiazepine

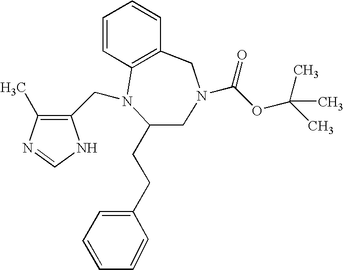

4-(1,1-Dimethylethoxycarbonyl)-2-phenylethyl-2,3,4,5-tetrahydrobenzodiazepine.

To a stirred solution of 2-phenylethyl-2,3,4,5-tetrahydrobenzodiazepine (700 mg, 2.8 mmol) in THF at rt was added di-tert-butyldicarbonate (670 mg, 3.1 mmol), and the mixture was allowed to stir at rt for 18 h. The solvent was removed, and the residue was purified by silica gel column chromatography (hexanes/EtOAc, 3:1) to give Compound A as a clear oil (800 mg, 81%). ¹H NMR (CDCl₃) δ1.40 (s, 9H), 1.72 (m, 2H), 2.75 (m, 2H), 3.0 (m, 1H), 3.40 (m, 1H), 3.70 (d, 1H), 3.80 (d, 1H), 4.40 (dd, 2H), 6.64 (m, 1H), 6.86 (m, 1H), 7.20 (m, 7H). ¹³C NMR (CDCl₃) δ28.37, 32.62, 35.02, 51.38, 53.21, 57.07, 79.90, 120.77, 126.03, 127.87, 128.33, 129.50, 14.32, 147.47, 155.18.

4-(1,1-Dimethylethoxycarbonyl)-1-(4-methylimidazol-5-ylmethyl)-2-phenylethyl-2,3,4,5-tetrahydrobenzodiazepine.

The title compound was prepared by following the same procedure as employed for the preparation of 4-(4-fluorophenylsulfonyl)-1-(4-methylimidazol-5-ylmethyl)-2-phenylethyl-2,3,4,5-tetrahydrobenzodiazepine, except 4-(1,1-dimethylethoxycarbonyl)-2-phenylethyl-2,3,4,5-tetrahydrobenzodiazepine was used as substrate. The title compound (Example 8) was obtained as a solid. MS: 447 (M+H).

### EXAMPLE 10

### 1-(Imidazol-4-ylmethyl)-2-phenylethyl-4-phenylsulfonyl-2,3,4,5-tetrahydrobenzodiazepine.

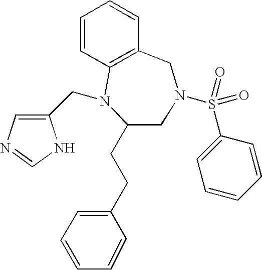

4-(9-Fluorenylmethoxycarbonyl)-2-phenylethyl-2,3,4,5-tetrahydrobenzodiazepine.

To a stirred solution of 2-phenylethyl-2,3,4,5-tetrahydrobenzodiazepine (110 mg, 0.44 mmol) in CH₂Cl₂ and saturated sodium bicarbonate solution at rt was added 9-fluorenylmethyl chloroformate (120 mg, 0.45 mmol), and the mixture was allowed to stir at rt for 18 h. The organic layer was then separated, dried, and concentrated to give Compound A as an oil, which was used directly in the next step.

4-(9-Fluorenylmethoxycarbonyl)-1-(imidazol-4-ylmethyl)-2-phenylethyl-2,3,4,5-tetrahydrobenzodiazepine.

The title compound was prepared by following the same procedure as employed for preparing 4-(4-fluorophenylsulfonyl)-1-(4-methylimidazol-5-ylmethyl)-2-phenylethyl-2,3,4,5-tetrahydrobenzodiazepine, except 4-(9-fluorenylmethoxycarbonyl)-2-phenylethyl-2,3,4,5-tetrahydrobenzodiazepine and imidazolealdehyde were used as substrates.

1-(Imidazol-4-ylmethyl)-2-phenylethyl-4-phenylsulfonyl-2,3,4,5-tetrahydrobenzodiazepine trifluoroacetate.

The title compound was prepared by a solid phase synthetic method from 4-(9-fluorenylmethoxycarbonyl)-1-(imidazol-4-ylmethyl)-2-phenylethyl-2,3,4,5-tetrahydrobenzodiazepine. 2-Chlorotrityl resin (purchased from Nova Biochem, 01-64-0021, 200 mg) was suspended in DMF (2 mL) for 10 min, then 4-(9-fluorenylmethoxycarbonyl)-1-(imidazol-4-ylmethyl)-2-phenylethyl-2,3,4,5-tetrahydrobenzodiazepine (244 mg) was added, and the mixture was shaken for 18 h. The mixture was filtered, and the resin was washed sequentially with DMF, MeOH, and CH₂Cl₂. The resin was then dried in a vacuum oven for 18 h, and then suspended in DMF. Piperidine (1 mL) was added, and the mixture was shaken for 48 h. The resin was again washed sequentially with DMF, MeOH, and CH₂Cl₂, and then dried. The resin was then again suspended in DMF; diisopropylethylamine and benzenesulfonyl chloride were added sequentially, and the mixture was shaken for 18 h. After successive washings as before, the dried resin was suspended in CH₂Cl₂, and then TFA and triethylsilane were added sequentially. The mixture was then shaken for 18 h and filtered. The filtrate was concentrated, and the residue was purified by reverse phase HPLC using water, MeOH, and a small amount of TFA as eluents. The desired fractions were collected and lyophilized to give the title compound as a white solid (100 mg). MS: 472 (M+H).

### EXAMPLES 11-36

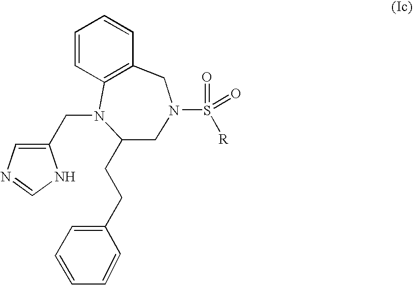

Compounds having the formula (Ic) wherein R has the values listed in Table 3 were prepared by following the same procedure as described for the preparation of 1-Imidazol-4-ylmethyl)-2-phenylethyl-4-phenylsulfonyl-2,3,4,5-tetrahydrobenzodiazepine trifluoroacetate (step C of Example 4), except different sulfonyl chlorides were used in step C.

TABLE 3

| Example # | Structure (R) | Characterization |
|---|---|---|
| 11 | H₃C— | MS 411 |
| | | [M + H]⁺ |
| 12 | 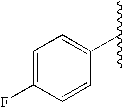 | MS 491 [M + H]⁺ |
| 13 | 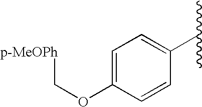 | MS 609 [M + H]⁺ |
| 14 | 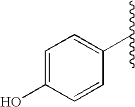 | MS 489 [M + H]⁺ |
| 15 | 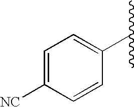 | MS 498 [M + H]⁺ |
| 16 | 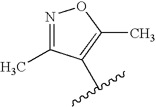 | MS 492 [M + H]⁺ |
| 17 | 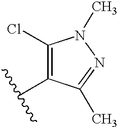 | MS 525 [M + H]⁺ |
| 18 | 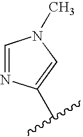 | MS 477 [M + H]⁺ |
| 19 | CF₃— | MS 465 |
| | | [M + H]⁺ |
| 20 | 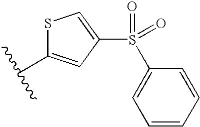 | MS 619 [M + H]⁺ |
| 21 | 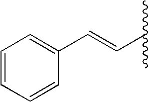 | MS 499 [M + H]⁺ |
| 22 | 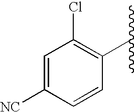 | MS 532 [M + H]⁺ |
| 23 | 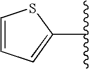 | MS 479 [M + H]⁺ |
| 24 | 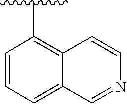 | MS 524 [M + H]⁺ |
| 25 | 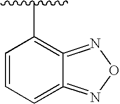 | MS 515 [M + H]⁺ |
| 26 | 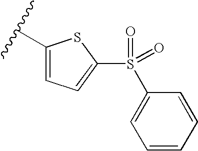 | MS 619 [M + H]⁺ |
| 27 | 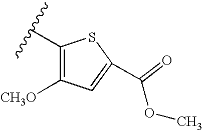 | MS 567 [M + H]⁺ |
| 28 | 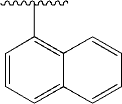 | MS 523 [M + H]⁺ |
| 29 | 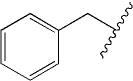 | MS 487 [M + H]⁺ |
| 30 | 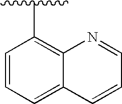 | MS 524 [M + H]⁺ |
| 31 | 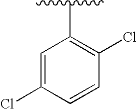 | MS 541 [M + H]⁺ |
| 32 | 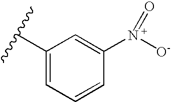 | MS 518 [M + H]⁺ |
| 33 | 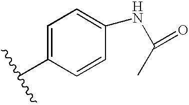 | MS 530 [M + H]⁺ |
| 34 | 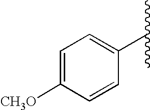 | MS 503 [M + H]⁺ |
| 35 | 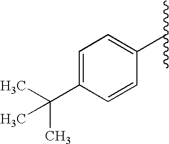 | MS 529 [M + H]⁺ |
| 36 | 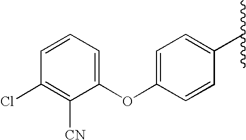 | MS 624 [M + H]⁺ |

### EXAMPLE 37

### 4-(4-tert-Butylphenylsulfonyl)-1-(imidazol-2-ylmethyl)-2-phenylethyl-2,3,4,5-tetrahydrobenzodiazepine

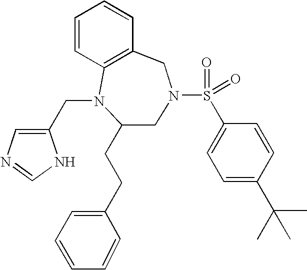

To a stirred solution of 4-(4-tert-butylphenylsulfonyl)-2-phenylethyl-2,3,4,5-tetrahydrobenzodiazepine (intermediate 7D) (85 mg, 0.19 mmol) in 1 mL of 1:1 1,2-dichloroethane and acetic acid was added imidazole-2-aldehyde (50 mg, 0.5 mmol, 2.5 equiv), and the mixture was allowed to stir at rt for 30 min. Sodium triacetoxyborohydride (120 mg, 0.56 mmol, 3.0 equiv) was then added, and the mixture was stirred overnight. Upon completion of the reaction, the reaction mixture was poured into EtOAc and ammonium hydroxide solution, and the organic layer was separated, dried, and concentrated to give the title compound as a solid (95 mg, 95%). MS 529 [M+H]⁺

### EXAMPLE 38

### 4-(4-tert-Butylphenylmethyl)-(imidazol-4-ylmethyl)-2-phenylethyl-2,3,4,5-tetrahydrobenzodiazepin-3-one

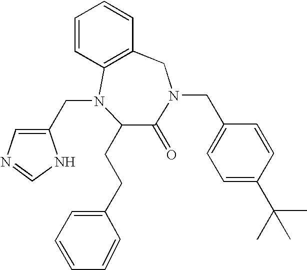

38A. 4-(4-tert-Butylbenzyl)-2-phenylethyl-2,3,4,5-tetrahydrobenzodiazepin-3-one.

To a solution of 2-phenylethyl-2,3,4,5-tetrahydrobenzodiazepin-3-one (intermediate 1B) (348 mg, 1.31 mmol) in 4 mL DMF in a 50-mL round-bottomed flask at 0° C. was added sodium hydride (60% dispersion in mineral oil, 52 mg, 1 equiv). After 1 h at 0° C., 4-tert-butylbenzyl bromide (252 μL, 1.38 mmol, 1.05 equiv) was added and the mixture was allowed to warm to rt overnight. The reaction mixture was then partitioned between water and EtOAc, and washed with 5% LiCl solution (2×5 mL). The organic layer was dried (Na₂SO₄), and concentrated under reduced pressure. Purification by flash chromatography (silica gel, hexanes/EtOAc gradient) afforded the desired product as a yellow crystalline solid. MS 413 [M+H]⁺

38B. 4-(4-tert-Butylphenylmethyl)-1-(imidazol-4-ylmethyl)-2-phenylethyl-2,3,4,5-tetrahydrobenzodiazepin-3-one.

The title compound was prepared using General Procedure A listed for Example 1, Step E, except imidazole-4-aldehyde was used. MS 479 [M+H]⁺

### EXAMPLE 39

### 4-(4-tert-Butylphenylmethyl)-1-(4-methylimidazol-5-ylmethyl)-2-phenylethyl-2,3,4,5-tetrahydrobenzodiazepin-3-one

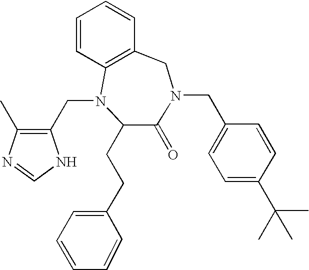

The title compound was prepared in a manner similar to that used for the compound of Example 38 from intermediate 38A to give the desired product. MS 493 [M+H].⁺

1. A compound having the formula (I), 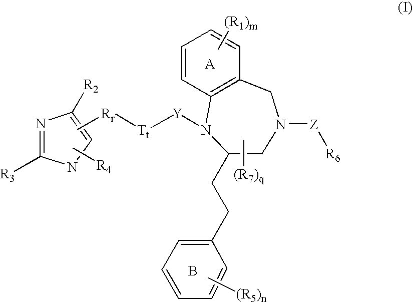
or a stereoisomer, a pharmaceutically-acceptable salt, hydrate, or prodrug thereof, wherein:
R₁ and R₅ are attached to any available carbon atom of phenyl rings A and B, respectively, and at each occurrence are independently selected from alkyl, substituted alkyl, halogen, cyano, nitro, OR₈, NR₈R₉, C(═O)R₈, CO₂R₈, C(═O)NR₈R₉, NR₈C(═O)R₉, NR₈C(═O)OR₉, S(O)₀R₉, NR₈SO₂R₉, SO₂NR₈R₉, cycloalkyl, heterocycle, aryl, and heteroaryl, and/or two of R₁ and/or two of R₅ join together to form a fused benzo ring;
R₂, R₃ and R₄ are independently selected from hydrogen, alkyl, and substituted alkyl, or one of R₂, R₃ and R₄ is a bond to R, T or Y and the other of R₂, R₃ and R₄ is selected from hydrogen, alkyl, and substituted alkyl;
Z and Y are independently selected from C(═O), —CO₂—, —SO₂—, —CH₂—, —CH₂C(═O)—, and —C(═O)C(═O)—, or Z may be absent;
R and T are selected from —CH₂—, —C(═O)—, and —CH[(CH₂)ₚ(Q)]—, wherein Q is NR₁₀R₁₁, OR₁₀ or CN;
R₆ is selected from alkyl, alkenyl, substituted alkyl, substituted alkenyl, aryl, cycloalkyl, heterocyclo, and heteroaryl; provided that where R₂ is hydrogen, Z-R₆ together are not —SO₂-Me or 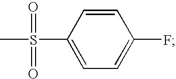
R₇ is selected from hydrogen, alkyl, substituted alkyl, alkenyl, substituted alkenyl, aminoalkyl, halogen, cyano, nitro, keto (═O), hydroxy, alkoxy, alkylthio, C(═O)H, acyl, CO₂H, alkoxycarbonyl, carbamyl, sulfonyl, sulfonamidyl, cycloalkyl, heterocycle, aryl, and heteroaryl;
R₈ and R₉ are independently selected from hydrogen, alkyl, substituted alkyl, cycloalkyl, heterocycle, aryl, and heteroaryl, or R₈ and R₉ taken together to form a heterocycle or heteroaryl, except R₉ is not hydrogen when attached to a sulfonyl group as in SO₂R₉;
R₁₀ and R₁₁ are independently selected from hydrogen, alkyl, and substituted alkyl;
m and n are independently selected from 0, 1, 2 and 3;
o, p and q are independently 0, 1 or 2; and
r and t are 0 or 1.

2. A compound according to claim 1, having the formula, 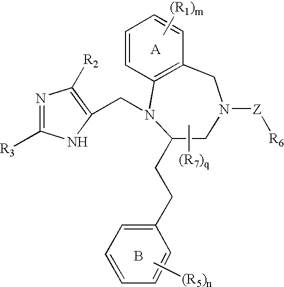
or a stereoisomer, a pharmaceutically-acceptable salt, hydrate, or prodrug thereof, in which:
R₁ and R₅ are attached to any available carbon atom of phenyl ring A and phenyl ring B, respectively, and at each occurrence are independently selected from C₁₋₆alkyl, substituted C₁₋₆alkyl, halogen, cyano, O(C₁₋₆alkyl), O(phenyl), O(benzyl), NH₂, NH(C₁₋₆alkyl), N(C₁₋₆alkyl)₂, C(═O)H, C(═O)(C₁₋₆alkyl), CO₂H, CO₂(C₁₋₆alkyl), C(═O)NH₂, C(═O)NH(C₁₋₆alkyl), C(═O)N(C₁₋₆alkyl)₂, NHC(═O)(C₁₋₆alkyl), S(O)₂(C₁₋₆alkyl), NHSO₂(C₁₋₆alkyl), SO₂NH₂, SO₂NH(C₁₋₆alkyl), SO₂N(C₁₋₆alkyl)₂, C₃₋₇cycloalkyl, phenyl, five or six membered heteroaryl, or four to seven membered heterocyclo, and/or two of R₁ and/or two of R₅ join together to form a fused benzo ring;
R₂ and R₃ are independently selected from hydrogen and C₁₋₄alkyl;
Z is —CO₂—, —SO₂—, or is absent;
R₆ is selected from optionally-substituted alkyl, alkenyl, aryl, and heteroaryl.
m and n are independently selected from 0, 1, and 2; and
q is 0 or 1.

3. A compound according to claim 1, or a stereoisomer, a pharmaceutically-acceptable salt, hydrate, or prodrug thereof, in which R₁ and R₅ are selected from hydrogen, C₁₋₄ alkyl, halogen, cyano, trifluoromethyl, trifluoromethoxy, and OC₁₋₄ alkyl.

4. A compound according to claim 1, or a stereoisomer, a pharmaceutically-acceptable salt, hydrate, or prodrug thereof, in which R₂, R₃ and R₄ are selected from hydrogen and C₁₋₄ alkyl.

5. A compound according to claim 1, or a stereoisomer, a pharmaceutically-acceptable salt, hydrate, or prodrug thereof, in which R₂ is C₁₋₄ alkyl.

6. A compound according to claim 1, or a stereoisomer, a pharmaceutically-acceptable salt, hydrate, or prodrug thereof, in which R and T are absent.

7. A compound according to claim 1, or a stereoisomer, a pharmaceutically-acceptable salt, hydrate, or prodrug thereof, in which Y is CH₂.

8. A compound according to claim 1, or a stereoisomer, a pharmaceutically-acceptable salt, hydrate, or prodrug thereof, in which Z is —CO₂—, —SO₂—, or is absent.

9. A compound according to claim 1 or a stereoisomer, a pharmaceutically-acceptable salt, hydrate, or prodrug thereof, in which:
R₆ is selected from:
a) C₁₋₄alkyl or C₁₋₄alkenyl optionally substituted with up to three of halogen, aryl and CO₂C₁₋₆alkyl;
b) phenyl optionally substituted with up to three R₁₂ and/or having fused thereto a benzo-ring or a five to six membered heteroaryl;
c) five to six membered heteraryl optionally substituted with up to two R₁₂, and each R₁₂ is independently selected from each other R₁₂ from C₁₋₆alkyl, halogen, nitro, cyano, hydroxy, alkoxy, NHC(═O)alkyl, —CO₂alkyl, —SO₂phenyl, five to six membered monocyclic heteroaryl, and phenyloxy or benzyloxy in turn optionally substituted with halogen, C₁₋₄alkyl, and/or O(C₁₋₄alkyl).

10. The compound of claim 1 or a stereoisomer, a pharmaceutically-acceptable salt, hydrate, or prodrug thereof, in which Z-R₆ taken together are selected from:
i. thiophenyl optionally substituted with R₁₄;
ii. imidazolyl optionally substituted with R₁₄;
iii. —CH(aryl)(CO₂C₁₋₆alkyl);
iv. —CO₂-alkyl;
v. —SO₂-alkyl optionally substituted with up to three of halogen and/or phenyl;
vi. —SO₂-alkenyl optionally substituted with phenyl; and
vii. 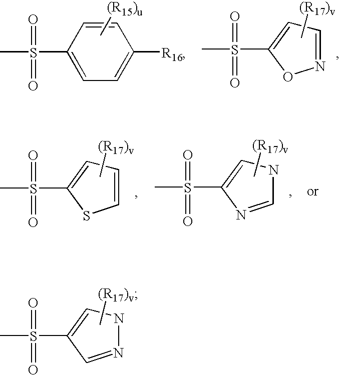
wherein
R₁₅ is halogen, alkyl, nitro, cyano, hydroxy, alkoxy, NHC(═O)alkyl, and/or two R₁₅ groups are taken together to form a fused benzo ring or a five to six membered heteroaryl;
R₁₆ is selected from hydrogen, halogen, alkyl, nitro, cyano, hydroxy, alkoxy, NHC(═O)alkyl, and phenyloxy or benzyloxy in turn optionally substituted with 1 to 3 of halogen, cyano, and C₁₋₄alkoxy;
R₁₇ is selected from alkyl, alkoxy, CO₂C₁₋₆alkyl, and SO₂phenyl; and
u and v are independently 0, 1 or 2.

11. A compound according to claim 1, having the formula, 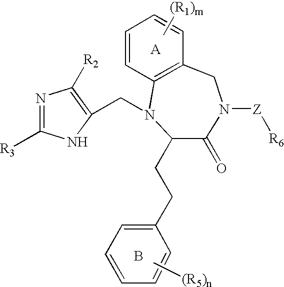
or a stereoisomer, a pharmaceutically-acceptable salt, hydrate, or prodrug thereof.

12. A compound having the formula, 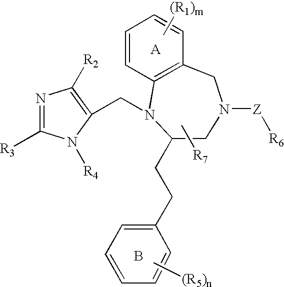
or a stereoisomer, a pharmaceutically-acceptable salt, hydrate, or prodrug thereof, wherein:
R₁ and R₅ are attached to any available carbon atom of phenyl ring A and phenyl ring B, respectively, and at each occurrence are independently selected from alkyl, substituted alkyl, halogen, cyano, nitro, hydroxy, alkoxy, alkylthio, alkylamino, C(═O)H, acyl, CO₂H, alkoxycarbonyl, carbamyl, sulfonyl, sulfonamidyl, cycloalkyl, heterocycle, aryl, and heteroaryl, and/or two of R₁ and/or two of R₅ join together to form a fused benzo ring;
R₂, R₃ and R₄ are independently selected from hydrogen and alkyl;
Z is —CO₂—, —SO₂—, or is absent;
R₆ is selected from:
a) C₁₋₄alkyl or C₁₋₄alkenyl optionally substituted with up to three of halogen, aryl and CO₂C₁₋₆alkyl;
b) phenyl optionally substituted with up to three R₁₂ and/or having fused thereto a benzo-ring or a five to six membered heteroaryl;
c) heteroaryl selected from thiophenyl, imidazolyl, pyrazolyl, and isoxazolyl, wherein said heteroaryl is optionally substituted with up to two R₁₂,
provided that where R₂ is hydrogen, Z-R₆ together are not —SO₂-Me or 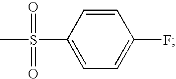
R₇ is selected from hydrogen, keto (═O), C₁₋₆alkyl, substituted C₁₋₆alkyl, halogen, cyano, O(C₁₋₆alkyl), O(phenyl), O(benzyl), NH₂, NH(C₁₋₆alkyl), N(C₁₋₆alkyl)₂, C(═O)H, C(═O)(C₁₋₆alkyl), CO₂H, CO₂(C₁₋₆alkyl);
R₁₂ at each occurrence is independently selected from each other R₁₂ from the group consisting of C₁₋₆alkyl, halogen, nitro, cyano, hydroxy, alkoxy, NHC(═O)alkyl, —CO₂alkyl, —SO₂phenyl, five to six membered monocyclic heteroaryl, and phenyloxy or benzyloxy in turn optionally substituted with halogen, C₁₋₄alkyl, and/or O(C₁₋₄alkyl); and
m and n are independently selected from 0, 1, or 2.

13. A compound according to claim 12 or a stereoisomer, a pharmaceutically-acceptable salt, hydrate, or prodrug thereof, wherein:
Z is —SO₂—;
R₆ is selected from C₁₋₄alkyl, trifluoromethyl, benzyl, C₂₋₃alkenyl substituted with phenyl, 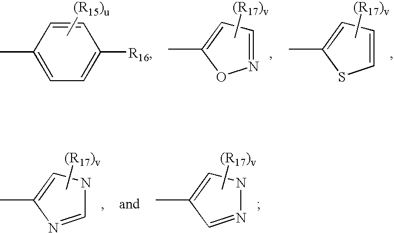
R₁₅ is halogen, alkyl, nitro, cyano, hydroxy, alkoxy, NHC(═O)alkyl, and/or two R₁₅ groups are taken together to form a fused benzo ring or a five to six membered heteroaryl;
R₁₆ is selected from hydrogen, halogen, alkyl, nitro, cyano, hydroxy, alkoxy, NHC(═O)alkyl, and phenyloxy or benzyloxy in turn optionally substituted with 1 to 3 of halogen, cyano, and C₁₋₄alkoxy;
R₁₇ is selected from alkyl, alkoxy, CO₂C₁₋₆alkyl, and SO₂phenyl; and
u and v are independently 0, 1 or 2.

14. A pharmaceutical composition comprising at least one compound of claim 1 and a pharmaceutically-acceptable carrier or diluent.

15. A pharmaceutical composition comprising at least one compound of claim 12 and a pharmaceutically-acceptable carrier or diluent.

16. The pharmaceutical composition of claim 14 further comprising at least one other therapeutic agent selected from one or more of potassium channel openers, calcium channel blockers, sodium hydrogen exchanger inhibitors, antiarrhythmic agents, antiatherosclerotic agents, anticoagulants, antithrombotic agents, prothrombolytic agents, fibrinogen antagonists, diuretics, antihypertensive agents, ATPase inhibitors, mineralocorticoid receptor antagonists, phospodiesterase inhibitors, antidiabetic agents, anti-inflammatory agents, antioxidants, angiogenesis modulators, antiosteoporosis agents, hormone replacement therapies, hormone receptor modulators, oral contraceptives, antiobesity agents, antidepressants, antianxiety agents, antipsychotic agents, antiproliferative agents, antitumor agents, antiulcer and gastroesophageal reflux disease agents, growth hormone agents and/or growth hormone secretagogues, thyroid mimetics, anti-infective agents, antiviral agents, antibacterial agents, antifungal agents, cholesterol/lipid lowering agents and lipid profile therapies, and agents that mimic ischemic preconditioning and/or myocardial stunning.

17. The pharmaceutical composition of claim 16 in which the at least one other therapeutic agent is selected from one or more of antiatherosclerotic agents, anticoagulants, antithrombotic agents, antihypertensive agents, and antidiabetic agents.

18. The pharmaceutical composition of claim 17 wherein the at least one other therapeutic agent is an antihypertensive agent selected from ACE inhibitors, AT-1 receptor antagonists, ET receptor antagonists, dual ET/AII receptor antagonists, and vasopepsidase inhibitors, or an antiplatelet agent selected from GPIIb/IIIa blockers, P2Y₁ and P2Y₁₂ antagonists, thromboxane receptor antagonists, and aspirin.

19. A method of treating a mithochondrial F₁F₀ ATP hydrolase associated disorder in a patient comprising administering to the patient in need of such treatment an effective amount of at least one compound having the formula (I), 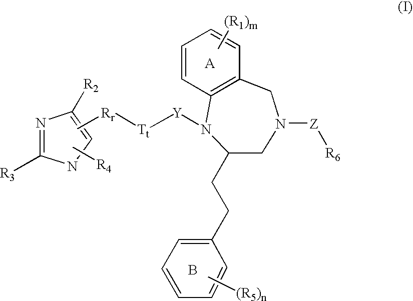
or a stereoisomer, a pharmaceutically-acceptable salt, hydrate, or prodrug thereof, wherein:
R₁ and R₅ are attached to any available carbon atom of phenyl rings A and B, respectively, and at each occurrence are independently selected from alkyl, substituted alkyl, halogen, cyano, nitro, OR₈, NR₈R₉, C(═O)R₈, CO₂R₈, C(═O)NR₈R₉, NR₈C(═O)R₉, NR₈C(═O)OR₉, S(O)₀R₉, NR₈SO₂R₉, SO₂NR₈R₉, cycloalkyl, heterocycle, aryl, and heteroaryl, and/or two of R₁ and/or two of R₅ join together to form a fused benzo ring;
R₂, R₃ and R₄ are independently selected from hydrogen, alkyl, and substituted alkyl, or one of R₂, R₃ and R₄ is a bond to R, T or Y and the other of R₂, R₃ and R₄ is selected from hydrogen, alkyl, and substituted alkyl;
Z and Y are independently selected from C(═O), —CO₂—, —SO₂—, —CH₂—, —CH₂C(═O)—, and —C(═O)C(═O)—, or Z may be absent;
R and T are selected from —CH₂—, —C(═O)—, and —CH[(CH₂)ₚ(Q)]—, wherein Q is NR₁₀R₁₁, OR₁₀ or CN;
R₆ is selected from alkyl, alkenyl, substituted alkyl, substituted alkenyl, aryl, cycloalkyl, heterocyclo, and heteroaryl;
R₇ is selected from hydrogen, alkyl, substituted alkyl, alkenyl, substituted alkenyl, aminoalkyl, halogen, cyano, nitro, keto (═O), hydroxy, alkoxy, alkylthio, C(═O)H, acyl, CO₂H, alkoxycarbonyl, carbamyl, sulfonyl, sulfonamidyl, cycloalkyl, heterocycle, aryl, and heteroaryl;
R₈ and R₉ are independently selected from hydrogen, alkyl, substituted alkyl, cycloalkyl, heterocycle, aryl, and heteroaryl, or R₈ and R₉ taken together to form a heterocycle or heteroaryl, except R₉ is not hydrogen when attached to a sulfonyl group as in SO₂R₉;
R₁₀ and R₁₁ are independently selected from hydrogen, alkyl, and substituted alkyl;
m and n are independently selected from 0, 1, 2 and 3;
o, p and q are independently 0, 1 or 2; and
r and t are 0 or 1.

20. The method of claim 19 wherein the mithochondrial F₁F₀ ATP hydrolase disorder is selected from myocardial infarction, ventricular hypertrophy, coronary artery disease, non-Q wave MI, congestive heart failure, cardiac arrhythmias, unstable angina, chronic stable angina, Prinzmetal's angina, high blood pressure, intermittent claudication, peripheral occlusive arterial disease, thrombotic or thromboembolic symptoms of thromboembolic stroke, venous thrombosis, arterial thrombosis, cerebral thrombosis, pulmonary embolism, cerebral embolism, thrombophilia, disseminated intravascular coagulation, restenosis, atrial fibrillation, ventricular enlargement, atherosclerotic vascular disease, atherosclerotic plaque rupture, atherosclerotic plaque formation, transplant atherosclerosis, vascular remodeling atherosclerosis, cancer, surgery, inflammation, systematic infection, artificial surfaces, interventional cardiology, immobility, medication, pregnancy and fetal loss, and diabetic complications comprising retinopathy, nephropathy and neuropathy.
